SEQUENCE LISTING (33 sequences)

SEQ ID NO: 1 - RNA, 19 nt, Homo sapiens
  source 1..19: mol_type = genomic RNA
gagctggtggcgtaggcaa

SEQ ID NO: 2 - RNA, 19 nt, Homo sapiens
  source 1..19: mol_type = genomic RNA
gagcttgtggcgtaggcaa

SEQ ID NO: 3 - RNA, 19 nt, Homo sapiens
  source 1..19: mol_type = genomic RNA
gagctgatggcgtaggcaa

SEQ ID NO: 4 - RNA, 19 nt, Homo sapiens
  source 1..19: mol_type = genomic RNA
gagctggtgacgtaggcaa

SEQ ID NO: 5 - RNA, 19 nt, synthetic construct
  source 1..19: mol_type = other RNA
ctcgaatactgcatccgtt

SEQ ID NO: 6 - RNA, 25 nt, synthetic construct
  source 1..25: mol_type = other RNA
ccttgacgatacagctaattcagaa

SEQ ID NO: 7 - RNA, 25 nt, synthetic construct
  source 1..25: mol_type = other RNA
ggatattctcgacacagcaggtcaa

SEQ ID NO: 8 - RNA, 25 nt, synthetic construct
  source 1..25: mol_type = other RNA
gcaggtcaagaggagtacagtgcaa

SEQ ID NO: 9 - RNA, 25 nt, synthetic construct
  source 1..25: mol_type = other RNA
gggctttctttgtgtatttgccata

SEQ ID NO: 10 - RNA, 25 nt, synthetic construct
  source 1..25: mol_type = other RNA
ggctttctttgtgtatttgccataa

SEQ ID NO: 11 - RNA, 25 nt, synthetic construct
  source 1..25: mol_type = other RNA
cgagaaattcgaaaacataaagaaa

SEQ ID NO: 12 - RNA, 25 nt, synthetic construct
  source 1..25: mol_type = other RNA
gggctatatttacatgctactaaat

SEQ ID NO: 13 - RNA, 25 nt, synthetic construct
  source 1..25: mol_type = other RNA
ggctatatttacatgctactaaatt

SEQ ID NO: 14 - RNA, 25 nt, synthetic construct
  source 1..25: mol_type = other RNA
gggacatatgcagtgtgatccagtt

SEQ ID NO: 15 - RNA, 25 nt, synthetic construct
  source 1..25: mol_type = other RNA
gcagtgtgatccagttgttttccat

SEQ ID NO: 16 - RNA, 25 nt, synthetic construct
  source 1..25: mol_type = other RNA
ggaatgttggtcatatcaaacatta

SEQ ID NO: 17 - RNA, 19 nt, synthetic construct
  source 1..19: mol_type = other RNA
gtggttcagtccttcacct

SEQ ID NO: 18 - RNA, 25 nt, synthetic construct
  source 1..25: mol_type = other RNA
gggagcttggaagacgatcagcaat

SEQ ID NO: 19 - RNA, 25 nt, synthetic construct
  source 1..25: mol_type = other RNA
cctactatgtgtgtggactggagta

SEQ ID NO: 20 - RNA, 25 nt, synthetic construct
  source 1..25: mol_type = other RNA
ggagatgttgcagtaaagatcctaa

SEQ ID NO: 21 - RNA, 19 nt, synthetic construct
  source 1..19: mol_type = other RNA
ggatttcgatgtcagactt

SEQ ID NO: 22 - RNA, 19 nt, synthetic construct
  source 1..19: mol_type = other RNA
gcactgtagcaccaaagta

SEQ ID NO: 23 - RNA, 19 nt, synthetic construct
  source 1..19: mol_type = other RNA
gcaccaaagtacctactat

SEQ ID NO: 24 - RNA, 19 nt, synthetic construct
  source 1..19: mol_type = other RNA
ggtacatgacaaaggacaa

SEQ ID NO: 25 - RNA, 19 nt, synthetic construct
  source 1..19: mol_type = other RNA
gctcagggaatggactatt

SEQ ID NO: 26 - RNA, 19 nt, synthetic construct
  source 1..19: mol_type = other RNA
gggaatggactatttgcat

SEQ ID NO: 27 - RNA, 19 nt, synthetic construct
  source 1..19: mol_type = other RNA
ggagattttggtttggcaa

SEQ ID NO: 28 - RNA, 19 nt, synthetic construct
  source 1..19: mol_type = other RNA
cccagatcctgtcttccat

SEQ ID NO: 29 - RNA, 19 nt, synthetic construct
  source 1..19: mol_type = other RNA
ggagcctgctttggtacta

SEQ ID NO: 30 - RNA, 19 nt, synthetic construct
  source 1..19: mol_type = other RNA
gggttttaattttgttttt

SEQ ID NO: 31 - RNA, 19 nt, synthetic construct
  source 1..19: mol_type = other RNA
gggttttaattttgttttt

SEQ ID NO: 32 - protein, 17 aa, synthetic construct
KHHHKHHHKHHHKHHHK

SEQ ID NO: 33 - protein, 18 aa, synthetic construct
KHHHKHHHHKHHHKHHHK

(19) United States
(12) Patent Application Publication
(10) Pub. No.: US 2024/0141353 A1
(43) Pub. Date: May 2, 2024

(54) SIRNAS AGAINST KRAS AND RAF1

(71) Applicants: David M. EVANS, Germantown, MD (US); Vera SIMONENKO, Germantown, MD (US)

(72) Inventors: David M. EVANS, Germantown, MD (US); Vera SIMONENKO, Germantown, MD (US)

(21) Appl. No.: 18/495,687

(22) Filed: Oct. 26, 2023

Related U.S. Application Data
(60) Provisional application No. 63/419,360, filed on Oct. 26, 2022.

(51) Int. Cl. C12N 15/113; A61K 9/00; A61K 9/51; A61P 35/00

(52) U.S. Cl. CPC C12N 15/1135; A61K 9/0019; A61K 9/5169; A61P 35/00; C12N 2310/14

(57) ABSTRACT

siRNA molecules and pharmaceutical compositions containing siRNA molecules are provided for inhibiting expression of KRAS, RAF1 and mutant KRAS peptides or proteins. Methods of treatment of cancer are provided in which the pharmaceutical compositions are administered to a subject in need thereof. The cancer may be lung, colon, or pancreatic cancers, including non-small cell lung cancer (NSCLC). Combinations of siRNAs, packaged in nanoparticles with co-polymer carriers and delivered simultaneously to target cells, elicit an additive or synergistic effect to inhibit tumorous cell growth.

### CROSS-REFERENCE TO RELATED APPLICATIONS

This application claims priority to U.S. Provisional Patent Application Ser. No. 63/419,360, filed Oct. 26, 2022, the disclosure of which is hereby incorporated by reference in its entirety.

### SEQUENCE LISTING

The instant application contains a Sequence Listing submitted electronically in ST.26 (XML) format and hereby incorporated by reference in its entirety. The XML file, created on Sep. 1, 2023, is named 4690.0062I_SL and is 29 kilobytes in size.

### FIELD

siRNA agents, pharmaceutical compositions and methods of their delivery are provided for inhibiting expression of KRAS peptides or proteins, including mutated KRAS peptides and proteins, in lung and pancreatic cancers.

### BACKGROUND

Kirsten rat sarcoma viral oncogene homolog (“KRAS”) has emerged as an important oncogenic driver with therapeutic potential in several forms of cancer. KRAS mutations are commonly found in solid tumors, particularly in pancreatic, colon, and lung cancers such as non-small cell lung cancer (NSCLC), a heterogeneous disease that is difficult to treat. These cancers require continued activation and KRAS signaling, making KRAS a potential target for therapy (Salgia et al., Cell Reports Medicine 2, 100186, (2021)). KRAS is a small protein and lacks sufficient binding pockets accessible for interaction with small molecule drugs. For those NSCLC patients who are KRAS-mutant, the current treatment is chemotherapy with an average survival rate of only 22 months (Id.). Sotorasib is an inhibitor that specifically targets the G12C mutation in KRAS and is approved to treat NSCLC.

RAF1 is a MAP kinase kinase kinase (MAP3K), which functions downstream of the Ras family of membrane associated GTPases to which it binds directly. Activated RAF1 phosphorylates MEK1 and MEK2, which in turn phosphorylate and activate ERK1 and ERK2. Activated ERKs play an important role in the control of gene expression involved in the cell division cycle, apoptosis, cell differentiation and cell migration.

Double-stranded RNA has been shown to silence gene expression via RNA interference (RNAi). Short-interfering RNA (siRNA)-induced RNAi regulation shows great potential to treat a wide variety of human diseases from cancer to other traditional undruggable disease. RNA interference can be induced for inhibiting RAF and KRAS-mutant peptides or proteins, or other regulatory intermediates that promote the progression of a variety of cancers.

### BRIEF DESCRIPTION OF THE FIGURES

FIG. 1 shows a list of the siRNA molecules evaluated against their targets in NSCLC cells.

FIG. 2(a) shows the results of screening synthetic siRNAs against KRAS in the A549 lung cancer cell line.

FIG. 2(b) shows the results of screening several siRNAs against KRAS on cell viability in the A549 lung cancer cell line.

FIG. 2(c) shows the results of screening synthetic siRNAs against KRAS in the A549 lung cancer cell line.

FIG. 2(d) shows the results of screening several siRNAs against RAF1 on cell viability in the A549 lung cancer cell line.

FIG. 3 shows the synergistic effect of the combination of KRAS(#1) and RAF1(#2) siRNA molecules (SEQ ID Nos. 6 and 19) and a non-silencing (NS) control on cell viability in the MIA PaCa (KRAS G12C) pancreatic cancer cell line.

FIG. 4 shows the synergistic effect of the combination of KRAS(#1) and RAF1(#2) siRNA molecules (SEQ ID Nos. 6 and 19) on cell viability in A549 (KRAS G12S) cells, with data normalized to non-silencing (NS) RNA.

FIG. 5(a) shows the synergistic effect of the combination of KRAS(#1) and RAF1(#2) siRNA molecules (SEQ ID Nos. 6 and 19) on cell viability in H358 (KRAS G12C) cells.

FIG. 5(b) shows the effect of the combination of KRAS(#1) and RAF1(#2) siRNA molecules (SEQ ID Nos. 6 and 19) on cell viability in H1299 (KRAS WT) cells.

FIG. 5(c) shows the additive effect of the combination of KRAS(#1) and RAF1(#2) siRNA molecules (SEQ ID Nos. 6 and 19) on cell viability in A427 (KRAS G12D) cells.

FIG. 5(d) shows the effect of the combination of KRAS(#1) and RAF1(#2) siRNA molecules (SEQ ID Nos. 6 and 19) on cell viability in NL20 normal lung cells with data normalized to NS-RNA.

### SUMMARY

siRNA agents, pharmaceutical compositions and methods of their delivery are provided for treating lung, colon, and pancreatic cancers by reducing the expression of KRAS and RAF1, including certain mutant forms of KRAS, as well as other regulatory intermediates. Specific embodiments include siRNA molecules containing a double stranded (duplex) oligonucleotide, that targets a complementary nucleotide sequence in a single stranded (ss) target RNA molecule, such as a peptide or protein that may promote the progression of cancer. These siRNA molecules include several against KRAS and common KRAS mutations (including anti-mutant KRAS at the 12^{th} and 13^{th} codons, e.g., KRAS G12C, KRAS G12D), as well as siRNAs against RAF which provide a synergistic effect when delivered simultaneously with the KRAS siRNA(s) in KRAS mutant cells.

Also provided are pharmaceutical compositions containing one or more of these siRNA molecules (each or the combination) packaged in nanoparticles along with a co-polymer carrier comprising histidine and lysine, such as HKP, HKP(+H), H3K4b, and/or H3K4b(+H).

In some embodiments, the siRNA sequence(s) are capable of inhibiting RAF1 when delivered simultaneously with a KRAS siRNA in the same nanoparticles. The pharmaceutical composition may contain a single siRNA molecule and histidine polypeptide (co-polymer) carrier packaged in nanoparticles; in some embodiments, these siRNA molecules may include KRAS #1, and/or RAF1 #2 (SEQ ID Nos. 6 and 19, respectively).

In some embodiments, the pharmaceutical composition comprises two siRNA molecules packaged together in the same nanoparticle, such as KRAS #1 and RAF1(#2).

Also provided are methods of treating a variety of cancers such as lung cancer, including NSCLC, colon and pancreatic cancer, reducing or inhibiting tumor growth. These methods involve administering a therapeutically effective amount of a pharmaceutical composition containing one or more of the siRNA molecules and a co-polymer carrier, and where the target mRNA encoding a peptide or protein of interest comprising one or more KRAS mutations such as KRAS G12C, KRAS G12D, KRAS G12S and KRAS G13D. In some embodiments the carrier is a branched histidine-containing polypeptide such as H3K4b or H3K4b(+H). The target mRNA encodes a peptide or protein such as KRAS and/or RAF1, where the combination of siRNA molecules creates an additive or synergistic effect to reduce or inhibit tumor growth.

The subject that is treated may be a mammal, for example a human.

### DETAILED DESCRIPTION

Small interfering RNA molecules (siRNAs), their combinations, pharmaceutical compositions and methods of use are provided. siRNA target genes include Kirsten rat sarcoma viral oncogene homolog (“KRAS”), and/or RAF1. The disclosed pharmaceutical compositions comprise these siRNA combinations and histidine-lysine copolymer carriers, mixed and formed into nanoparticles. Disclosed methods involve treating subjects having lung, pancreatic or other cancers. When an siRNA targeting the KRAS gene is combined with an siRNA targeting RAF1, a synergistic or additive effect often can be observed, providing an enhanced therapeutic benefit in mammals, including humans. Specifically, potent siRNA sequences are provided that silence regions of KRAS in combination with RAF1 that are identical or nearly identical in humans, mice and non-human primates. The double stranded (ds) siRNA molecule or other nucleic acid may have a length of 19 to 27 base pairs of nucleotides; 20 to 30 base pairs; or 24 to 28 base pairs. The molecule may have blunt ends at both ends or may have overhangs at one or both ends.

The siRNA molecule may include a chemical modification at the individual nucleotide level or at the oligonucleotide backbone level (discussed further infra), or it may have no modifications. In one embodiment a KRAS siRNA possesses strand lengths of 25 nucleotides. In other embodiments, a KRAS and/or RAF1 siRNA may have strand lengths of 19 to 25 nucleotides, not including 3′ terminal overhangs such as dTdT. The strands may have dTdT overhangs on one or both strands. When an siRNA is 19 nucleotides long, each strand advantageously further contains a dTdT overhang at the 3′ end of both strands. The dTdT overhang is not counted in determining the strand length. The siRNA molecules can be asymmetric where one strand is shorter than the other (typically by 2 bases e.g. a 21mer with a 23mer or a 19mer with a 21mer or a 23mer with a 25mer).

Methods are provided for inducing inhibition of two targets to obtain a therapeutic response in treatment of cancer by simultaneously targeting KRAS and RAF1 by dual inhibition using siRNAs co-delivered together to the same cell via a nanoparticle mediated delivery system—advantageously comprising a branched histidine lysine polypeptide nanoparticle (H3K4b or H3K4b(+H)). The skilled artisan will recognize that siRNAs against other targets may also be included in the nanoparticles.

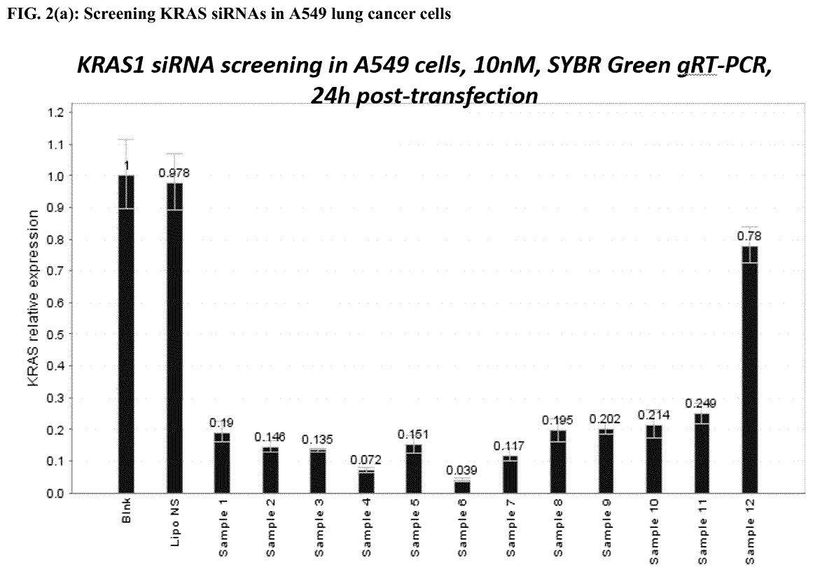

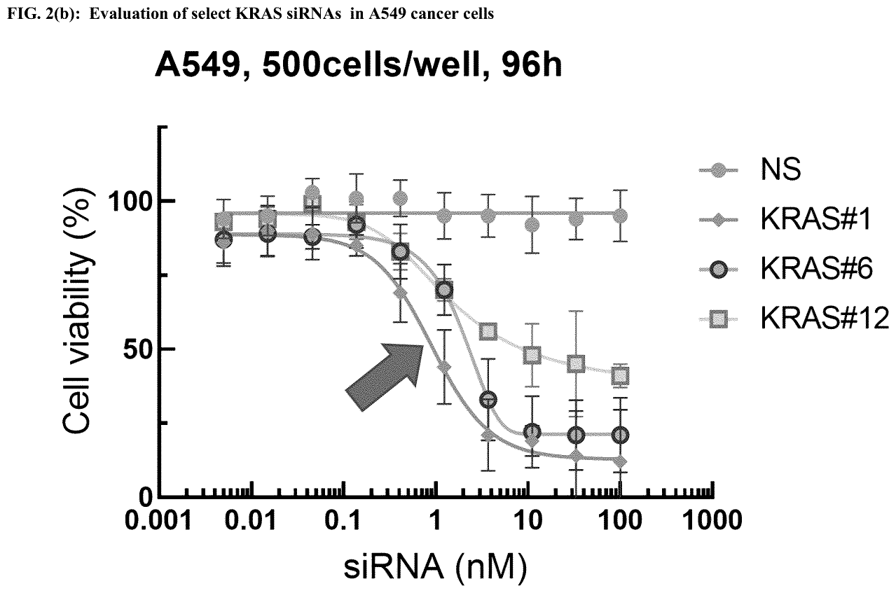

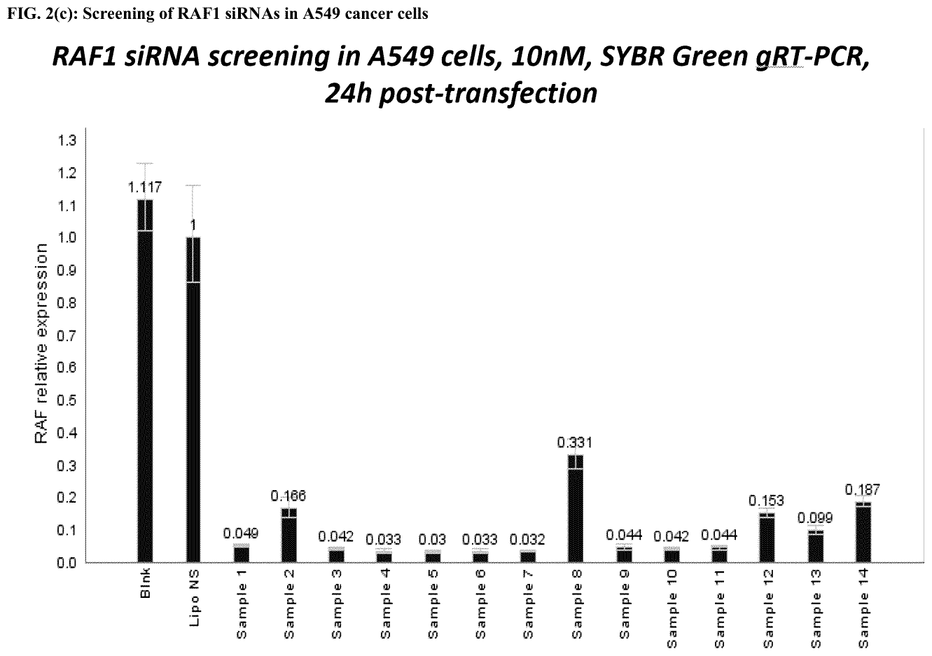

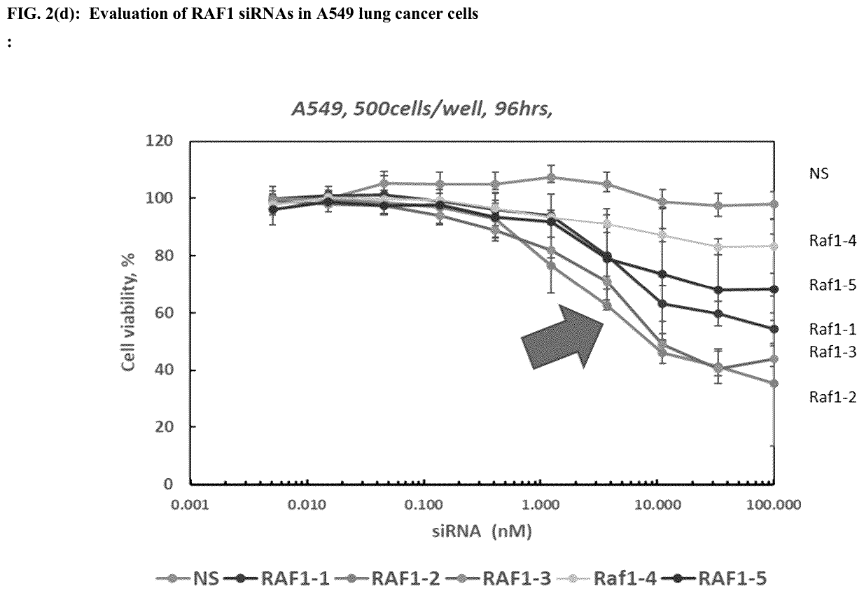

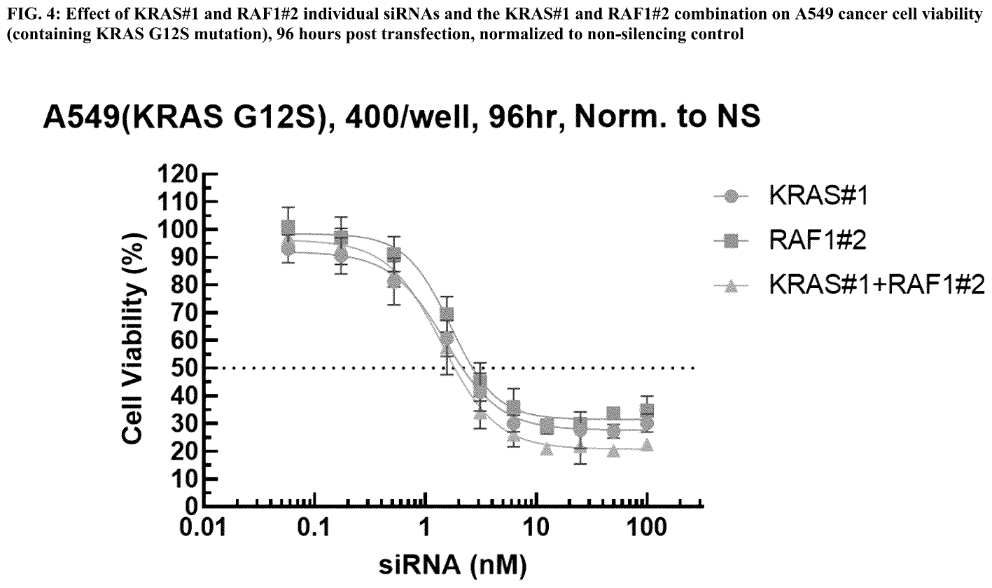

Multiple siRNAs were screened to identify the most potent sequence (most sequences not shown). The KRAS gene sequences in mice and humans were compared for regions of identity where siRNAs would be predicted to induce optimal silencing. Several combinations of peptides and proteins that may act synergistically to promote the progression of lung cancer (e.g., NSCLC) were identified in the literature, and siRNA therapeutics were designed to target combinations of these peptides and proteins. These synthetic siRNA molecules were screened and evaluated for their efficacy not only as individual agents but in combination with other siRNAs for their synergism against multiple genes, one of which was KRAS or RAF1. Results of the evaluation of these individual siRNA molecules and their combinations are shown in FIGS. 2-5 and described in more detail in the examples below.

A histidine-lysine copolymer carrier providing a nanoparticle delivery agent advantageously is used to deliver these combinations of two or more siRNAs to the appropriate tissue in the body and into the same cell at the same time. The siRNAs are mixed with a copolymer delivery vehicle such as HKP, or with more branched carriers such as H3K4b or H3K4b(+H) so that the siRNA combination is packaged in the same nanoparticle. When delivered to cells in vitro or in vivo, the nanoparticles release the siRNAs simultaneously, silencing the dual targets. Targeting more than one peptide or protein in a tissue may provide an augmented, or synergistic effect to reduce or inhibit the growth of the tumor cells compared to that produced in many cases by either siRNA alone.

Specifically, an siRNA targeting KRAS or RAF1 or a combination was formulated within a nanoparticle and delivered to the tumor cell. These combinations were evaluated in vitro in pancreatic, colon, and lung cancer cells containing various KRAS mutations, and also in normal lung cells.

### KRAS and RAF

KRAS is primarily involved in the cellular responses to extracellular signals and has emerged as an important oncogenic driver with therapeutic potential in several forms of cancer. It plays a role in the down regulation of mitogen-activated protein kinase (MAPK/ERK) (MEK)/ERK and phosphoinositide-3-kinase/v-akt murine thymoma viral oncogene (PI3K/AKT) pathways (Sideris, et al., Anticancer Res. 39:533-540 (2019).

KRAS mutations are commonly found in solid tumors, particularly in pancreatic, colon, and lung cancer such non-small cell lung cancer (NSCLC), a heterogeneous disease, which is difficult to treat. These cancers also are associated with the resistance to MEK inhibitors targeting the RAS pathway. Mutant KRAS promotes down-regulation of MAPK or PI3K/AKT resulting in excessive cell proliferation and carcinogensis. KRAS mutations refer to a frequent alteration of guanine to adenine (G>C), most frequently found in codons 12 and 13 of the KRAS gene. (Sideris, et al., 2019). However, other mutations in KRAS also can be found, e.g., at codons 61 and 146 (Papke et al. ACS Pharmacology & Translational Science 4:703-712, (2021)).

Raf family proteins act as an activator of the mitogen-activated protein kinase (MAPK)/ERK kinase (MEK) pathway and an effector of Ras, and plays a role in cellular processes such as proliferation, differentiation, cell death and survival, metabolism and motility. Huang et al., Oncotarget, 8:68329-68337 (2017). The mammalian Raf kinase family includes A-Raf, B-Raf, and Raf1 (C-Raf). Raf1 is an effector of Ras, and activated Raf can phosphorylate MEK, which then activates ERK to regulate various cell processes.

During tumorigenesis, Raf1 interacts with different proteins to allow cross-talk between signaling pathways. Id. Raf1 activity is controlled by, inter alia, Protein phosphatase 2A (PP2A)/PP1. Other proteins that interact with Raf1 include p21-activated kinase (PAK3), serine/threonine kinase 3 (STK3), and protein kinase C (PKC). Id.

Designing siRNAs Against KRAS Mutations

Mutated KRAS is a predominant oncogene in lung cancer, with codons 12, 13, 61 and 146 having primary importance. The KRAS mutations most commonly found in NSCLC are G12C and G12D. Papke et al., (supra) identified these and other mutations in the 12^{th} and 13^{th} codons and designed an siRNA molecule (EFTX-D1, SEQ ID No. 5) that targets the KRAS wild type and KRAS mutant specific sequences. The sequences of the relevant portion of the wild-type (WT) and mutant KRAS sequences are shown below:

KRAS WT:
(SEQ ID No. 1)
GAGCUGGUGGCGUAGGCAA
G12C mutant:
(SEQ ID No. 2)
GAGCUUGUGGCGUAGGCAA
KRAS G12D mutant:
(SEQ ID No. 3)
GAGCUGAUGGCGUAGGCAA
KRAS G13D mutant):
(SEQ ID No. 4)
GAGCUGGUGACGUAGGCAA
EFTX-DI
(SEQ ID NO. 5)
CUCGAAUACUGCAUCCGUU

A number of anti-KRAS siRNAs were designed (SEQ ID Nos. 6-17) and one (KRAS(#1), SEQ ID No. 6) was identified as particularly potent in the A549 cancer cell line containing the KRAS G12S mutation. KRAS #1 was more potent than EFTX-D1 against the mutant mRNAs (see Example 3 below). Unlike EFTX-D1, KRAS(#1) siRNA did not affect KRAS WT expression.

KRAS#1:
(SEQ ID No. 6)
5′-CCTTGACGATACAGCTAATTCAGAA-3′
KRAS#2:
(SEQ ID NO. 7)
5′-GGATATTCTCGACACAGCAGGTCAA-3′
KRAS#3:
(SEQ ID No. 8)
5′-GCAGGTCAAGAGGAGTACAGTGCAA-3′
KRAS#4:
(SEQ ID No. 9)
5′-GGGCTTTCTTTGTGTATTTGCCATA-3′
KRAS#5:
(SEQ ID No. 10)
5′-GGCTTTCTTTGTGTATTTGCCATAA-3′
KRAS#6:
(SEQ ID No. 11):
5′-CGAGAAATTCGAAAACATAAAGAAA-3′
KRAS#7:
(SEQ ID No. 12)
5′-GGGCTATATTTACATGCTACTAAAT-3′
KRAS#8:
(SEQ ID No. 13):
5′-GGCTATATTTACATGCTACTAAATT-3′
KRAS#9:
(SEQ ID No. 14)
5′-GGGACATATGCAGTGTGATCCAGTT-3
KRA#10:
(SEQ ID No. 15)
5′-GCAGTGTGATCCAGTTGTTTTCCAT-3
KRAS#11:
(SEQ ID No. 16)
5′-GGAATGTTGGTCATATCAAACATTA-3′
KRAS#12):
(SEQ ID No. 17)
5′-GUGGUUCAGUCCUUCACCU-3′

Design of siRNAs Against RAF1

siRNAs against RAP1 (SEQ ID Nos. 18-31) were design-led and screened, and a single siRNA (RAF1(#2) (SEQ ID No. 19) was selected for further evaluation.

Anti-RAF1 siRNA Sequences:

RAFI(#1)):
(SEQ ID No. 18)
5′-GGGAGCTTGGAAGACGATCAGCAAT-3′
RAFI(#2):
(SEQ ID No. 19)
5′-CCTACTATGTGTGTGGACTGGAGTA-3′
RAF1(#3)):
(SEQ ID No. 20)
5′-GGAGATGTTGCAGTAAAGATCCTAA-3′
RAFI(#4)):
(SEQ ID No. 21)
5′-GGATTTCGATGTCAGACTT-3′
(RAFI(#5)):
(SEQ ID No. 22)
5′-GCACTGTAGCACCAAAGTA-3′
RAFI(#6)):
(SEQ ID No. 23)
5′-GCACCAAAGTACCTACTAT-3′
RAFI(#7)):
(SEQ ID No. 24)
5′-GGTACATGACAAAGGACAA-3′
RAFI(#8)):
(SEQ ID No. 25)
5′-GCTCAGGGAATGGACTATT-3′
RAFI(#9)):
(SEQ ID No. 26)
5′-GGGAATGGACTATTTGCAT-3′
RAFI(#10)):
(SEQ ID No. 27)
5′-GGAGATTTTGGTTTGGCAA-3′
RAFI(#11)):
(SEQ ID No. 28)
5′-CCCAGATCCTGTCTTCCAT-3′
RAFI(#12)):
(SEQ ID No. 29)
5′-GGAGCCTGCTTTGGTACTA-3′
RAFI(#13)):
(SEQ ID NO. 30)
5′-GGGTTTTAATTTTGTTTTT-3′
RAFI(#14)):
(SEQ ID No. 31)
5′-GGGTTTTAATTTTGTTTTT-3′

Example 2 discusses the evaluation of RAF1(#2) siRNA in combination with KRAS(#1) in a variety of cancer cell lines and normal lung cells.

### Definitions

As used herein, “oligonucleotide” refers to a chemically modified or unmodified nucleic acid molecule (RNA or DNA) having a length of less than 100 nucleotides (for example less than 50, less than 30, or less than 25 nucleotides). An oligonucleotide can be any number of types of nucleic acids, including: an siRNA, a microRNA, an anti-microRNA, a microRNA mimic, a dsRNA, a ssRNA, an aptamer, or a triplex forming oligonucleotides.

As used herein, an “siRNA molecule” or “RNAi molecule” is a duplex oligonucleotide, that is a short, double-stranded polynucleotide, that interferes with the expression of a gene in a cell, after the molecule is introduced into the cell. For example, an siRNA molecule targets and binds to a complementary nucleotide sequence in a single stranded target RNA molecule. By convention, when an siRNA molecule is identified by a particular nucleotide sequence, the sequence refers to the sense strand of the duplex molecule. One or more of the ribonucleotides comprising the molecule can be chemically modified by techniques known in the art. In addition to being modified at the level of one or more of its individual nucleotides, the backbone of the oligonucleotide can be modified. Additional modifications include the use of small molecules (e.g. sugar molecules), amino acids, peptides, cholesterol, and other large molecules for conjugation onto the siRNA molecule.

“Inhibition of expression” refers to the absence or significantly decreasing in the level of protein and/or mRNA product from the target gene. In some embodiments, the inhibition is complete following administration of a pharmaceutical composition comprising one or more siRNAs. In many embodiments inhibition is partial, but sufficient to detect or observe. Inhibition may be measured by determining a decrease in the level of mRNA and/or protein product from a target nucleic acid relative to a cell lacking the siRNA molecule, and maybe as little as 10%, 50%, or maybe complete, i.e., 100% inhibition. The effects of inhibition may be detectable quantitatively and/or qualitatively in the cell or organism.

siRNA molecules can directly target the activity of genes with minimum off-target events. By “off target events” it is meant that expression of nucleic acids are not inhibited by the siRNA molecules other than the target are decreased significantly. In the case of lung and pancreatic cancers, this offers a unique opportunity to address the unmet clinical treatment needs.

As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphorodithioates, phosphoramidates, methyl phosphonates, chiral-methyl phosphonates, 2′-O-methyl ribonucleotides, 2′-Fluoro ribonucleotides, peptide-nucleic acids (PNAs) and unlocked nucleic acids (UNAs; see, e.g., Jensen et al., Nucleic Acids Symposium Series 52: 133-4 (2008)), and derivatives thereof.

As used herein, “nucleotide” is used as recognized in the art to include those with natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other, see, e.g., Usman and McSwiggen, supra; Eckstein, et al., WO 92/07065; Usman et al, WO 93/15187; Uhlman & Peyman, supra. There are several examples of modified nucleic acid bases known in the art as summarized by Limbach, et al., Nucleic Acids Res. 22:2183, (1994). Examples of base modifications that can be introduced into nucleic acid molecules include, hypoxanthine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin, et al., Biochemistry 35:14090, 1996; Uhlman & Peyman, supra). A modified base indicates a nucleotide base other than adenine, guanine, cytosine and uracil at the 1′ position or their equivalents.

As used herein, a “modified nucleotide” or “modified residue” refers to a nucleotide having one or more modifications, typically non-naturally occurring modifications, to the nucleoside, the base, pentose ring, or phosphate group, although modifications may include naturally occurring modifications produced by enzymes that modify nucleotides, such as methyltransferases. Non-naturally occurring modifications in nucleotides include those with 2′ modifications, e.g., 2′-methoxy (2′-OMe), 2′-methoxyethoxy, 2′-fluoro (2′-F), 2′-allyl, 2′-O-[2-(methylamino)-2-oxoethyl], 4′-thio, 4′-CH₂—O-2′-bridge, 4′-(CH₂)2-O-2′-bridge, 2′-LNA or other bicyclic or “bridged” nucleoside analog, and 2′-O—(N-methylcarbamate) or those comprising base analogs.

As used herein, an “amino modification” means 2′-NH₂ or 2′-O—NH₂, which can be further modified, or be unmodified. Such modified groups are described, e.g., in Eckstein et al., U.S. Pat. No. 5,672,695 and Matulic-Adamic et al., U.S. Pat. No. 6,248,878. “Modified nucleotides” as used herein also can include nucleotide analogs as described above.

### Modifications

The double strand (ds) siRNA may be unmodified or chemically modified using modifications that are well known in the art. For example, one or more of the RNA nucleotides may be modified at the 2′ position with 2′-F or 2′-OMe, and/or at the 5′-position with —P(O)₂═S, —P(S)₂=0. Examples of chemical modifications include, without limitation, phosphorothioates, phosphorodithioates, phosphoramidates, methyl phosphonates, chiral-methyl phosphonates, 2′-O-methyl ribonucleotides, 2′-Fluoro ribonucleotides, peptide-nucleic acids (PNAs) and unlocked nucleic acids (UNAs; see, e.g., Jensen et al., Nucleic Acids Symposium Series 52: 133-4), and derivatives thereof. In other embodiments, the siRNA may also be modified by pegylation or lipid functionalization to improve the overall stability and bioavailability of the RNAi.

In certain embodiments, modifications may exist upon these agents in patterns on one or both strands of the double stranded ribonucleic acid (dsRNA). As used herein, modification at “alternating positions” indicates that every other nucleotide is a modified nucleotide or there is an unmodified nucleotide (e.g., an unmodified ribonucleotide) between every modified nucleotide over a defined length of a strand of the dsRNA (e.g., 5′-MNMNMN-3′; 3′-MNMNMN-5′; where M is a modified nucleotide and N is an unmodified nucleotide). The modification pattern starts from the first nucleotide position at either the 5′ or 3′ terminus according to a position numbering convention. The pattern of modified nucleotides at alternating positions may run the full length of the strand, but in certain embodiments includes at least 4, 6, 8, 10, 12, 14 nucleotides containing at least 2, 3, 4, 5, 6 or 7 modified nucleotides, respectively. Modifications with alternating pairs of positions indicates a pattern where two consecutive modified nucleotides are separated by two consecutive unmodified nucleotides over a defined length of a strand of the dsRNA (e.g., 5′-MMNNMMNNMMNN-3′; 3′-MMNNMMNNMMNN-5′; where M is a modified nucleotide and N is an unmodified nucleotide). The modification pattern starts from the first nucleotide position at either the 5′ or 3′ terminus according to a position numbering convention such as those described herein. The pattern of modified nucleotides at alternating positions may run the full length of the strand, but preferably includes at least 8, 12, 16, 20, 24, 28 nucleotides containing at least 4, 6, 8, 10, 12 or 14 modified nucleotides, respectively. These modification patterns are exemplary, and the skilled artisan will recognize that additional patterns may be used.

In certain embodiments, the first and second oligonucleotide sequences of the siRNA exist on separate oligonucleotide strands that can be and typically are chemically synthesized. In some embodiments, both strands contain 19 nucleotides, or both strands contain 25 nucleotides. These molecules may be completely complementary and have blunt ends, or may have dTdT overhangs on one or both strands. In certain embodiments the siRNA strands have differing lengths, with one possessing a blunt end at the 3′ terminus of a first strand (sense strand) and a 3′ overhang at the 3′ terminus of a second strand (antisense strand). The siRNA can also contain one or more deoxyribonucleic acid (DNA) base substitutions.

Suitable siRNA compositions that contain two separate oligonucleotides can be chemically linked outside their annealing region by chemical linking groups. Many suitable chemical linking groups are known in the art. Suitable groups will not block endonuclease activity on the siRNA and will not interfere with the directed destruction of the RNA transcribed from the target gene. Alternatively, the two separate oligonucleotides can be linked by a third oligonucleotide such that a hairpin structure is produced upon annealing of the two oligonucleotides making up the siRNA composition. The hairpin structure will not block endonuclease activity on the siRNA and will not interfere with the directed destruction of the target RNA.

The dsRNA molecules of the disclosed embodiments are added directly, or can be complexed with lipids (e.g., cationic lipids), packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers.

The selected siRNA molecules inhibit the expression of mutant KRAS nucleic acid sequences and related targets in lung, colon, and pancreatic cell lines. The siRNA molecules of the disclosed embodiments are capable of targeting multiple genes with a single effector sequence.

As described below in the Examples, these sequences were tested for the ability to inhibit gene expression in lung and pancreatic cell lines in vitro.

Formation of Nanoparticles Containing siRNAs Targeting KRAS and/or RAF1

The siRNA molecules containing the molecules described above advantageously are formulated into nanoparticles for administration to a subject. Various methods of nanoparticle formation are well known in the art. See, for example, Babu et al., IEEE Trans Nanobioscience, 15: 849-863 (2016). The nanoparticles may contain one or more lipids, including neutral and cationic lipids.

Advantageously, the nanoparticles are formed using one or more histidine/lysine (HKP) copolymers. Suitable HKP copolymers are described in WO/2001/047496, WO/2003/090719, and WO/2006/060182, and U.S. Pat. Nos. 7,163,695, 7,070,807, and 7,772,201, where the latter describes highly-branched HKP copolymers, which advantageously may be used in the disclosed embodiments. HKP copolymers form a nanoparticle containing an siRNA molecule, typically 60-400 nm in diameter. HKP and HKP(+H) both have a lysine backbone (three lysine residues) where the lysine side chain ε-amino groups and the N-terminus are coupled to [KH₃]₄K (SEQ ID NO: 32) (for HKP) or KH₃KH₄[KH₃]₂K (SEQ ID NO: 33) (for HKP(+H). The branched HKP carriers can be synthesized by methods that are well-known in the art including, for example, solid-phase peptide synthesis.

Methods of forming nanoparticles are well known in the art. Babu et al., supra. Advantageously, nanoparticles may be formed using a microfluidic mixer system, in which an siRNA targeting KRAS and/or RAF1 is mixed with one or more siRNAs targeting other proteins and the two siRNAs are mixed together before being formulated with HKP polymers at a fixed flow rate to give nanoparticles of a given size. The flow rate can be varied to vary the size of the nanoparticles produced. A suitable microfluidic mixer is, for example, a NanoAssemblr microfluidic instrument (Precision NanoSystems, Inc.).

Determination of Efficacy of the siRNA Molecules

Depending on the particular target mRNA sequences and the dose of the nanoparticle composition delivered, partial or complete loss of function for the KRAS, RAF1 and or other target mRNAs may be observed. A reduction or loss of mRNA levels, gene expression or encoded polypeptide expression in at least 10%, at least 20%, at least 30%, at least 40%, at least 50%, at least 60%, at least 70%, at least 80%, at least 90%, at least 95% or at least 99% or more of targeted cells is exemplary. Inhibition of targeted mRNA levels or gene expression refers to the absence (or observable decrease) in the level of the target mRNA or the RNA-encoded peptide or protein. Specificity refers to the ability to inhibit the target mRNA without manifesting effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

Inhibition of target mRNA sequence(s) by the dsRNA agents in the disclosed embodiments also can be measured based upon the effect of administration of such dsRNA agents upon development/progression of a target mRNA-associated disease or disorder, e.g., tumor formation, growth, metastasis, etc., either in vivo or in vitro. Treatment and/or reductions in tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., 10-fold, 100-fold, 1000-fold, 10⁵-fold, 10⁶-fold, or 10⁷-fold reduction in cancer cell levels could be achieved via administration of the nanoparticle composition to cells, a tissue, or a subject. The subject may be a mammal, such as a human.

### Determination of Dosage and Toxicity

Toxicity and therapeutic efficacy of the compositions may be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds advantageously exhibit high therapeutic indices.

Data from cell culture assays and animal studies can be used in formulating a range of dosage for use in humans. The dosage of the compositions advantageously is within a range of circulating concentrations that include the ED₅₀ with little or no toxicity. The dosage may vary within this range depending upon the dosage form employed and the route of administration utilized. For the compositions described herein, a therapeutically effective dose can be estimated initially from cell culture assays. A dose may be formulated in animal models to achieve a circulating plasma concentration range that includes the IC₅₀ (i.e., the concentration of the composition which achieves a half-maximal inhibition of symptoms) as determined in cell culture. Such information can be used to more accurately determine useful doses in humans. Levels in plasma may be measured, for example, by high performance liquid chromatography.

A therapeutically effective amount of a composition as described herein can be in the range of approximately 1 pg to 1000 mg. For example, 10, 30, 100, or 1000 pg, or 10, 30, 100, or 1000 ng, or 10, 30, 100, or 1000 μg, or 10, 30, 100, or 1000 mg, or 1-5 g of the compositions can be administered. In general, a suitable dosage unit of the compositions described herein will be in the range of 0.001 to 250 milligrams per kilogram body weight of the recipient per day, or in the range of 0.01 to 20 micrograms per kilogram body weight per day, or in the range of 0.001 to 5 micrograms per kilogram of body weight per day, or in the range of 1 to 500 nanograms per kilogram of body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. The pharmaceutical composition can be administered once daily, or may be dosed in dosage units containing two, three, four, five, six or more sub-doses administered at appropriate intervals throughout the day. In that case, the dsRNA contained in each sub-dose must be correspondingly smaller in order to achieve the total daily dosage unit. The dosage unit can also be compounded for a single dose over several days, e.g., using a conventional sustained release formulation which provides sustained and consistent release of the dsRNA over a period of several days. Sustained release formulations are well known in the art. In this embodiment, the dosage unit contains a corresponding multiple of the daily dose. Regardless of the formulation, the pharmaceutical composition must contain dsRNA in a quantity sufficient to inhibit expression of the target gene in the animal or human being treated. The composition can be compounded in such a way that the sum of the multiple units of dsRNA together contain a sufficient dose.

The compositions may be administered once, one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition as described herein may include a single treatment or, advantageously, can include a series of treatments.

As used herein, a pharmacologically or therapeutically effective amount refers to that amount of an siRNA composition effective to produce the intended pharmacological, therapeutic or preventive result. The phrases “pharmacologically effective amount” and “therapeutically effective amount” or “effective amount” refer to that amount of the composition effective to produce the intended pharmacological, therapeutic or preventive result. For example, if a given clinical treatment is considered effective when there is at least a 30 percent reduction in a measurable parameter associated with a disease or disorder, a therapeutically effective amount of a drug for the treatment of that disease or disorder is the amount necessary to effect at least a 30 percent reduction in that parameter.

### Pharmaceutical Compositions and Methods of Administration

The nanoparticle compositions may be further formulated as a pharmaceutical composition using methods that are well known in the art. The composition may be formulated to be compatible with its intended route of administration. Examples of routes of administration include parenteral, e.g., intravenous, intradermal, subcutaneous, oral (e.g., inhalation), transdermal (topical), transmucosal, and rectal administration. Advantageously, the pharmaceutical compositions are administered by intravenous, intradermal, or subcutaneous infusion or injection.

For transmucosal or transdermal administration, penetrants appropriate to the barrier to be permeated are used in the formulation. Such penetrants are generally known in the art, and include, for example, for transmucosal administration, detergents, bile salts and fusidic acid derivatives. Transmucosal administration can be accomplished for example, by using nasal sprays or suppositories. For transdermal administration, the active compounds are formulated into ointments, salves, gels, or creams, and applied through dermal patches and the lie, as generally known in the art.

The siRNA formulations can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996). Further, the siRNA formulations can also be administered by a method suitable for administration of nucleic acid agents, such as a DNA vaccine. These methods include gene guns, bio-injectors, and skin patches as well as needle-free methods such as the micro-particle DNA vaccine technology disclosed in the U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al (1998), Clin. Immunol. Immunopath., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that it may be administered through a syringe or similar device. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, using a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, trehalose, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The compositions may also be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

### Methods of Treatment

The compositions described herein may be used to treat proliferative diseases, such as cancer, characterized by expression, and particularly altered expression, of KRAS and/or RAF1. Exemplary cancers include: hepatocellular carcinoma, esophageal cancer, head and neck cancer, bladder cancer, pancreatic cancer, cholangiocarcinoma, lung cancer (NSCLC, SCLC, LUSC), colon cancer, glioblastoma, breast cancer, gastric adenocarcinomas, prostate cancer, ovarian carcinoma, cervical cancer, AML, ALL, myeloma or non-Hodgkins lymphoma. In these methods the composition may be delivered systemically or intratumorally.

Other cancers include renal cancer (e.g., papillary renal carcinoma), stomach cancer, medulloblastoma, thyroid carcinoma, rhabdomyosarcoma, osteosarcoma, squamous cell carcinoma (e.g., oral squamous cell carcinoma), melanoma, and hematopoietic disorders (e.g., leukemias and lymphomas, and other immune cell-related disorders). Further cancers include bladder, cervical (uterine), endometrial (uterine), head and neck, and oropharyngeal cancers.

The compositions may be administered as described above and, advantageously, may be delivered systemically or intratumorally. The compositions may be administered as a monotherapy, i.e. in the absence of another treatment, or may be administered as part of a combination regimen that includes one or more additional medications. Advantageously, the compositions are used as part of a combination regimen that includes an effective amount of at least one additional chemotherapy drug, as described below.

Further provided are methods of treating cancer in a subject, in which the nanoparticle composition as described above is administered together with an effective amount of a chemotherapy drug. Examples of suitable chemotherapy drugs include protein kinase inhibitors, platinum-containing drugs such as cisplatin, oxaloplatin, or carboplatin, docetaxel (Taxotere), gemcitabine (Gemzar), paclitaxel (Taxol), pemetrexed (Alimta), vinorelbine (Navelbine), Abraxane (Paclitaxel Albumin-stabilized Nanoparticle Formulation), Afatinib Dimaleate, Afinitor (Everolimus), Afinitor Disperz (Everolimus), Alecensa (Alectinib), Alectinib, Alimta (Pemetrexed Disodium), Alunbrig (Brigatinib), Atezolizumab, Avastin (Bevacizumab), Bevacizumab, Brigatinib, Capmatinib Hydrochloride, Carboplatin, Cemiplimab-rwlc, Ceritinib, Crizotinib, Cyramza (Ramucirumab), Dabrafenib Mesylate, Dacomitinib, Docetaxel, Doxorubicin Hydrochloride, Durvalumab, Entrectinib, Erlotinib Hydrochloride, Everolimus, Gavreto (Pralsetinib), Gefitinib, Gilotrif (Afatinib Dimaleate), Gemcitabine Hydrochloride, Gemzar (Gemcitabine Hydrochloride), Imfinzi (Durvalumab), Infugem (Gemcitabine Hydrochloride), Ipilimumab, Iressa (Gefitinib), Keytruda (Pembrolizumab), Libtayo (Cemiplimab-rwlc), Lorbrena (Lorlatinib), Lorlatinib, Mekinist (Trametinib Dimethyl Sulfoxide), Methotrexate Sodium, Mvasi (Bevacizumab), Necitumumab, Nivolumab, Osimertinib Mesylate, Paclitaxel, Paclitaxel Albumin-stabilized Nanoparticle Formulation, Paraplat (Carboplatin), Paraplatin (Carboplatin), Pembrolizumab, Pemetrexed Disodium, Portrazza (Necitumumab), Pralsetinib, Ramucirumab, Retevmo (Selpercatinib), Rozlytrek (Entrectinib), Selpercatinib, Nexavar (sorafenib) Tabrecta (Capmatinib Hydrochloride), Tafinlar (Dabrafenib Mesylate), Tagrisso (Osimertinib Mesylate), Tarceva (Erlotinib Hydrochloride), Taxotere (Docetaxel), Tecentriq (Atezolizumab), Tepmetko (Tepotinib Hydrochloride), Tepotinib Hydrochloride, Trametinib Dimethyl Sulfoxide, Trexall (Methotrexate Sodium), Vizimpro (Dacomitinib), Vinorelbine Tartrate, Xalkori (Crizotinib), Yervoy (Ipilimumab), Zirabev (Bevacizumab), Zykadia (Ceritinib), carboplatin-taxol, gemcitabine-cisplatin, Afinitor (Everolimus), Atezolizumab, Doxorubicin Hydrochloride, Durvalumab, Etopophos (Etoposide Phosphate), Etoposide, Etoposide Phosphate, Everolimus, Hycamtin (Topotecan Hydrochloride), Imfinzi (Durvalumab), Lurbinectedin, Methotrexate Sodium, Nivolumab, Opdivo (Nivolumab), Tecentriq (Atezolizumab), Topotecan Hydrochloride, and Trexall (Methotrexate Sodium).

The disclosed compositions and methods are further illustrated by the examples below, which are non-limiting.

### EXAMPLES

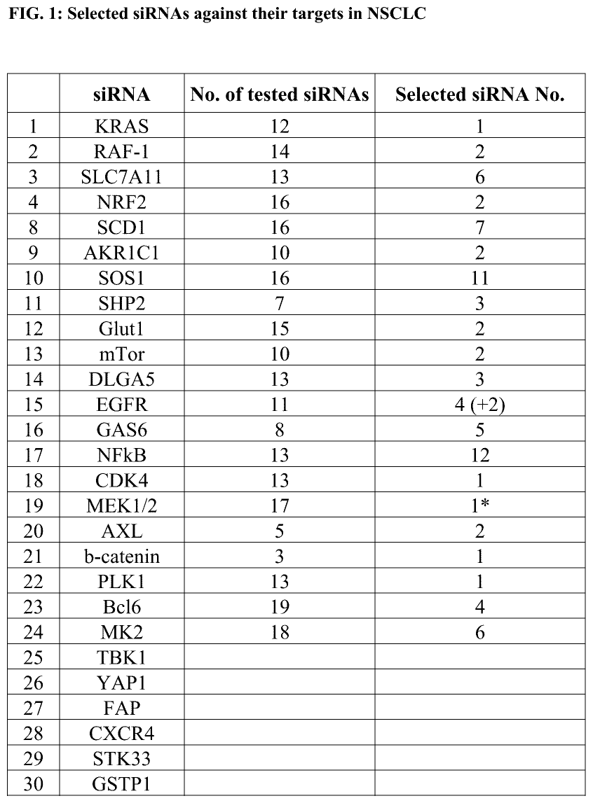

KRAS mutations have been detected in pancreatic, colon and lung cancers. siRNA molecules targeting KRAS, RAF1 and other genes were screened and evaluated for their efficacy not only as individual molecules but in combination for their synergism. See FIG. 1. Results of these evaluations of these individual siRNA molecules and combinations are shown in FIGS. 2-5.

### Example 1. Target Selection, and Design and Screening of Combinations of siRNA Molecules Acting Synergistically Against Target Combinations in Lung and Pancreatic Cancer

Screening and selection of siRNAs with high silencing activity were performed according to the following procedure:
1. All siRNA duplexes were pre-screened for knockdown effects in relevant cell lines with qRt-PCR end-point analysis. Western blot analysis was used to demonstrate specificity of mRNA inhibition with duplexes.
2. siRNA duplexes with the greatest ability to reduce gene expression were tested for the ability to kill cells.
3. The most potent sequences were used to identify combinations of siRNAs displaying synergistic effects on multiple targets in lung and pancreatic cancer cells.
4. An in vitro study was conducted, evaluating the siRNAs alone and in combination in some or all of the following human NSCLC cell lines: H1299 (KRAS wt), SK-MES (KRAS wt), H2030 (KRAS G12C), A549 (KRAS G12S), H358 (KRAS G12C), A427 (KRAS G12D), H460 (KRAS Q61H), and human bronchial epithelial cell line, NL20, derived from normal bronchial tissue. Some of the combinations were further evaluated in a pancreatic cancer cell line, MIA PaCa (G12C).

Sequences were chosen to obtain siRNA molecules that reduce expression of the identified KRAS or KRAS (codon 12 or 13)-mutated peptides or proteins, i.e., KRAS G12C, KRAS G12D, KRAS G12S, etc. siRNA molecules were also designed to be used against KRAS-mutated peptides or proteins in combination with other identified targets: KRAS signaling inhibitors such as RAF1, CHK1-AZ, ERK and MEK1. Other combinations of anti-KRAS sequences along with other siRNA molecules were also evaluated in lung and pancreatic cancer cells.

### Example 2. Screening and Evaluation of Anti-KRAS (KRAS #1, (SEQ ID No. 6) and Anti-RAF1(#2) (SEQ ID No. 19) siRNA Molecules Alone and in Combination

Twelve different anti-KRAS siRNA molecules (10 nM each) were evaluated in A549 lung cancer cells using SYBR Green qRT-PCR for 24 hours post transfection. FIGS. 2(a)-(b) show the screening results. KRAS1 siRNA samples 1-12 are shown to significantly reduce expression for various siRNAs targeting KRAS. KRAS #1 was selected as the most potent anti-KRAS siRNAs. Further, several anti-RAF1 siRNA molecules were evaluated in the A549 lung cancer cell line, and RAF1(#2) was chosen as most effective in the group of six (FIG. 2(c)-(d)).

### Evaluation Methodology

For cell viability studies, cells were dispensed at 400-500 cells per well in 96 well plates and siRNAs were transfected into cells on day 0 at varying concentrations. Cell viability was measured at 72-96 h post transfection by using Cell Titer Glo as per manufacturer's instructions (Promega). NL20 Cells (normal lung bronchoepithelial cells containing no KRAS mutation) were treated the same but were dispensed at 1000 cells per well. All values of cell inhibition were normalized to treatment with non-silencing siRNA as a control. In each experiment, the individual siRNAs were used at the concentration indicated and the dual treatment with siRNAs was used at the same final concentration where each individual siRNA was mixed at a 1:1 ratio.

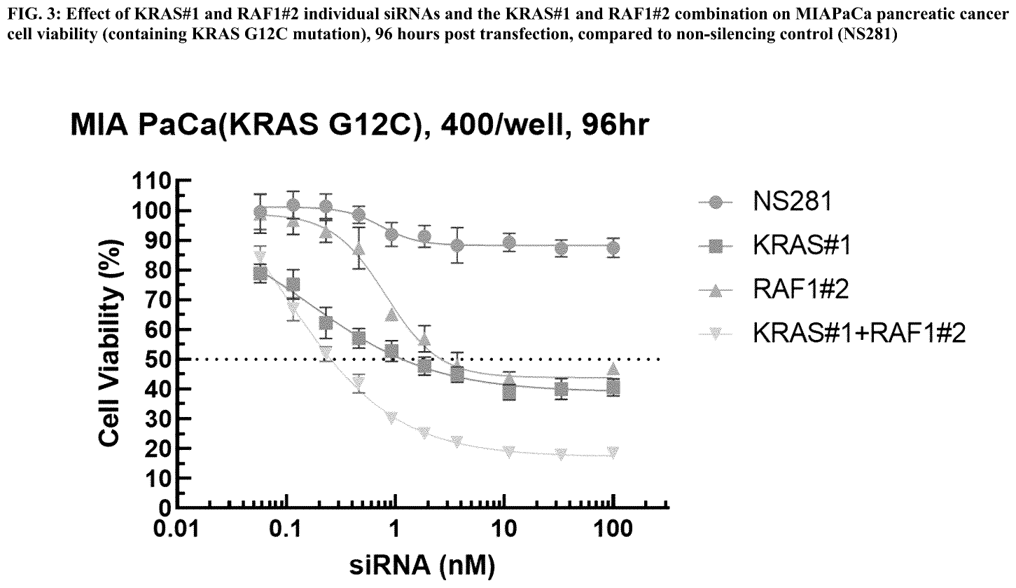

FIGS. 3-5 shows the effect of evaluating the individual KRAS #1 and RAF1 #2 siRNA molecules and the synergy of their combination in a variety of lung cancer and pancreatic cancer cell lines. A non-silencing RNA molecule was used as a control. In each of the cell types evaluated (H358, with the KRAS G12C mutation; A549, with the KRAS G12S mutation; H2030, with the KRAS G12C mutation; and H1299, KRAS wild type, MIA PaCa pancreatic cancer cells with the KRAS G12C mutation and in normal lung cells), the combination of KRAS #1 and RAF1 #2 exhibited the greatest and, in some cells (H358, MIA PaCa), the most profound, and at other times, equal effect (A549) to reduce cancer cell viability compared to one or both of the individual siRNAs. In NL20 (Normal WT KRAS) cells each siRNA was not effective in inhibiting cell viability and there was no additivity between the 2 siRNAs in effect. In H1299 lung cancer cells (WT KRAS) RAF1 #2 showed a greater effect than the combination which did not produce an IC₅₀ value at the highest concentration tested (100 nM).

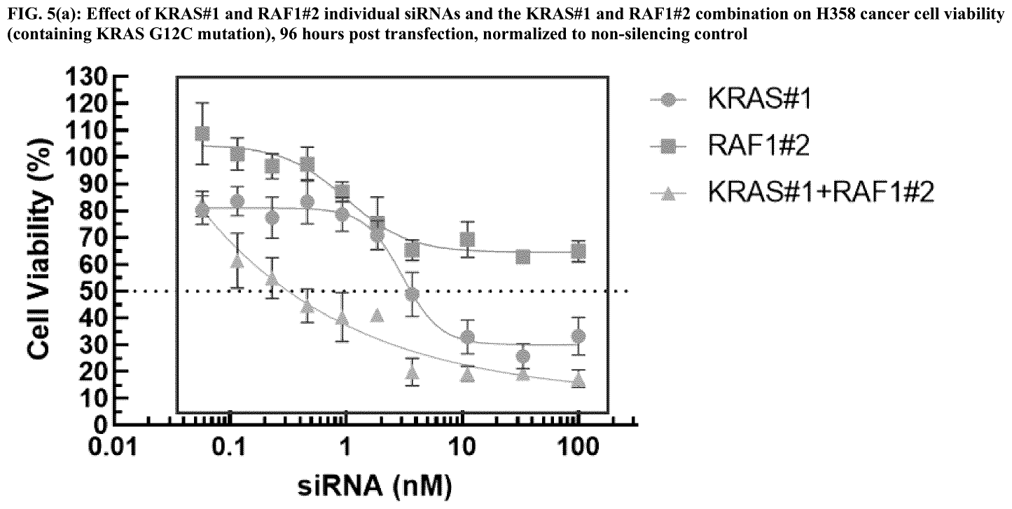

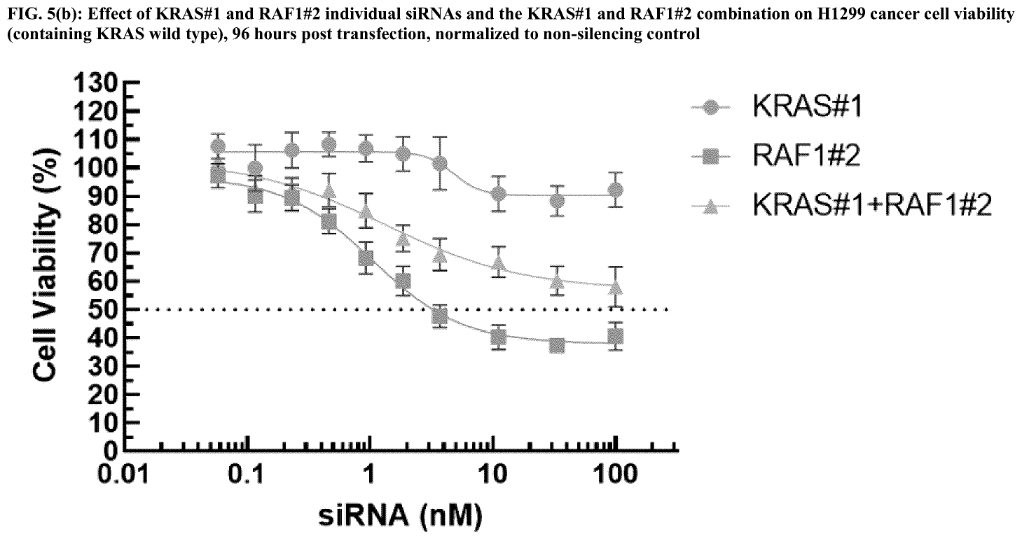

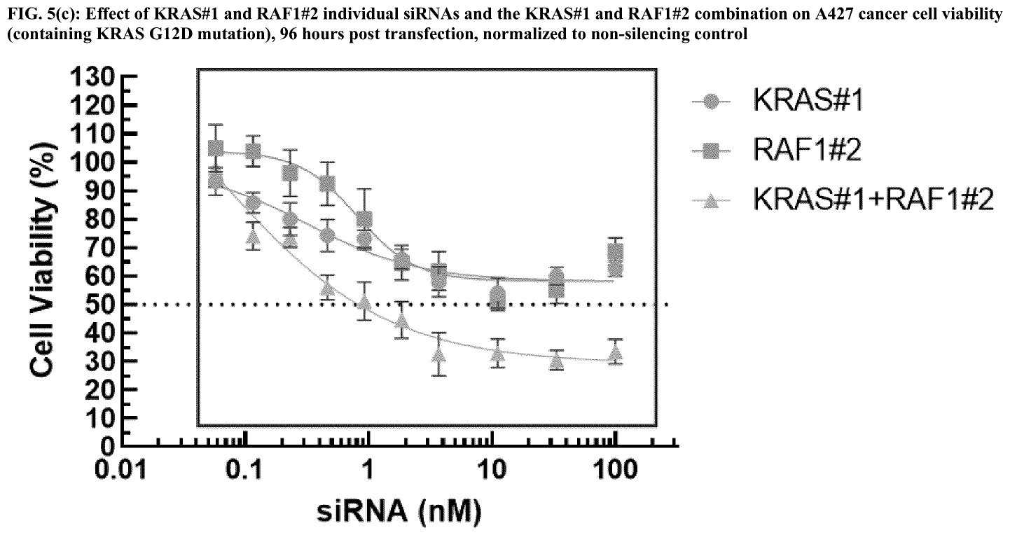

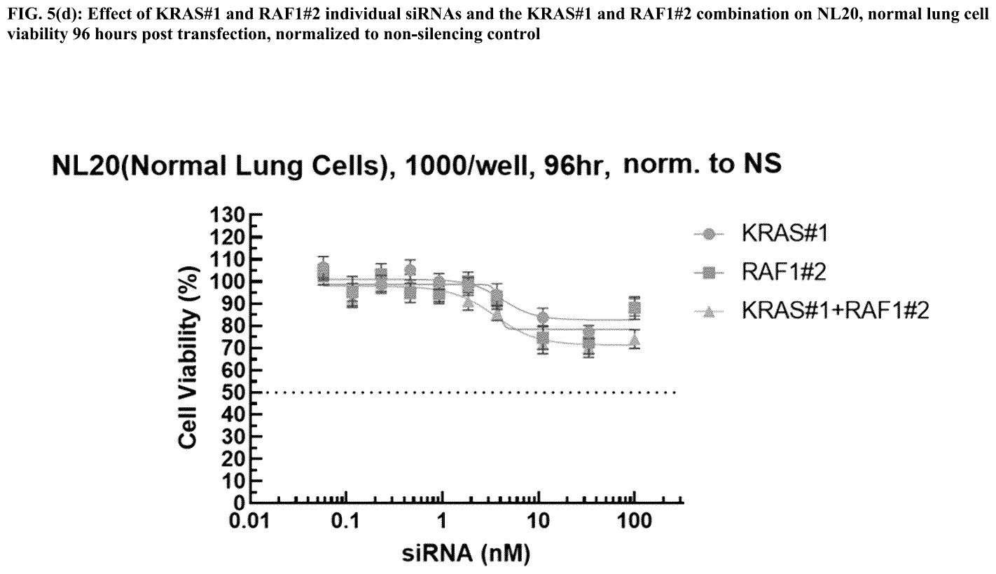

When the results were normalized to the non-silencing control (FIG. 5), the combination of KRAS #1 and RAF1(#2) also showed synergistic effects in the H358 (FIG. 5(a)) and another lung cancer cell line, A427 (FIG. 5(c)). Again, in H1299 (KRAS wt) cells, the combination did not produce an IC₅₀ at the highest concentration tested (100 nM) (FIG. 5(b)).

### Example 3. Evaluation of Anti-KRAS siRNA and KRAS Mutant-Specific siRNAs Along with Anti-RAF1(#2) (SEQ ID No. 28) siRNA Molecules Individually in Lung Cancer Cells

The same methodology was used as described in Example 2. The individual effects of anti-KRAS, anti-KRAS-mutant (G12C, G12D) and anti-KRAS wild type (WT) were evaluated in H358, H1299, and A427 cancer cell lines and in normal cell lines (NL20). The mutant anti-KRAS siRNAs was most effective in H358 (G12C mutation) cancer cells (data now shown) but did not have an effect in H1299 (KRAS WT) cancer cells, unlike the siRNA of Papke et al. In H358 cells containing the KRAS G12C mutation, the siRNA specific to the KRAS G12C mutation was most effective at about IC₅₀ of 1 nM, while KRAS(#1) siRNA exhibited an IC₅₀ of ˜2-3 nM.

Although this disclosure describes certain embodiments of the compositions and methods, and many details have been set forth for purposes of illustration, it will be apparent to those skilled in the art that these compositions and methods are susceptible to additional embodiments and that certain of the details described herein may be varied without departing from the basic principles of the disclosure.

We claim:

1. A small interfering RNA (siRNA) molecule comprising: a double stranded (duplex) oligonucleotide, wherein said oligonucleotide targets a complementary nucleotide sequence in a single stranded (ss) RNA molecule,
wherein said siRNA molecule comprises the molecule of SEQ ID NO:6, and
wherein said ss target RNA molecule encodes a KRAS or a mutant KRAS peptide or protein.

2. A small interfering RNA (siRNA) molecule comprising: a double stranded (duplex) oligonucleotide, wherein said oligonucleotide targets a complementary nucleotide sequence in a single stranded (ss) RNA molecule,
wherein said siRNA molecule comprises the molecule of SEQ ID NO:19, and
wherein said ss target RNA molecule encodes a RAF1 or a mutant RAF1 peptide or protein.

3. A composition comprising an siRNA molecule according to claim 1 and a second siRNA molecule, wherein said second molecule comprises a double stranded (duplex) oligonucleotide that targets a complementary nucleotide sequence in a single stranded (ss) RNA molecule,
wherein said siRNA molecule comprises the molecule of SEQ ID NO:19, and
wherein said ss target RNA molecule encodes a RAF1 or a mutant RAF1 peptide or protein.

4. A pharmaceutical composition comprising an siRNA molecule according to claim 1 and a co-polymer carrier, wherein the siRNA molecule and carrier are packaged as nanoparticles.

5. A pharmaceutical composition according to claim 3, further comprising a co-polymer carrier, wherein the siRNA molecules and carrier are packaged as nanoparticles.

6. The pharmaceutical composition according to claim 4, wherein the co-polymer carrier comprises a histidine-lysine copolymer

7. The composition according to claim 6, wherein said co-polymer is selected from the group consisting of HKP, H3K4b, HKP(+H), and H3K4b(+H).

8. The pharmaceutical composition according to claim 4, further comprising an siRNA molecule selected from the group consisting of SEQ ID Nos. 7-18 and 19-31.

9. A method of treating cancer in a subject, comprising administering to the subject a therapeutically effective amount of a pharmaceutical composition according to claim 4.

10. The method according to claim 9, wherein the cancer comprises cells having one or more KRAS mutations, and wherein administration of the pharmaceutical composition inhibits or reduces cancerous tumor growth in the subject.

11. The method according to claim 10, wherein said cells have a KRAS G12C, G12D and/or G12S mutation.

12. The method according to claim 9, wherein said cancer is selected from the group consisting of lung, colon, and pancreatic cancer.

13. The method of claim 12, wherein the cancer is NSCLC.

14. The method of claim 9, wherein the subject is a human.

15. The method according to claim 9, wherein said composition is administered systemically or intratumorally.

16. The method according to claim 16, wherein said composition is administered systemically via an intravenous or intraparenteral route.

17. A pharmaceutical composition comprising an siRNA molecule according to claim 2 and a co-polymer carrier, wherein the siRNA molecule and carrier are packaged as nanoparticles.

18. The pharmaceutical composition according to claim 17, wherein the co-polymer carrier comprises a histidine-lysine copolymer, wherein optionally said co-polymer is selected from the group consisting of HKP, H3K4b, HKP(+H), and H3K4b(+H).

19. The pharmaceutical composition according to claim 17, further comprising an siRNA molecule selected from the group consisting of SEQ ID Nos. 7-18 and 20-31.

20. A method of treating cancer in a subject, comprising administering to the subject a therapeutically effective amount of a pharmaceutical composition according to claim 3.

21. The method according to claim 20, wherein said cancer is selected from the group consisting of lung, colon, and pancreatic cancer.

22. The method of claim 21, wherein the cancer is NSCLC.

23. The method of claim 17, wherein the subject is a human.

24. The method according to claim 17, wherein said composition is administered systemically or intratumorally.

25. A method of treating cancer in a subject, comprising administering to the subject a therapeutically effective amount of a pharmaceutical composition according to claim 17.
